(19) United States
(12) Patent Application Publication
(10) Pub. No.: US 2006/0045855 A1
(43) Pub. Date: Mar. 2, 2006

(54) Oral composition for reducing plaque and microbial infections and whitening teeth

(76) Inventor: J. Alan Sasson, Brookline, MA (US)

Correspondence Address: BROMBERG & SUNSTEIN LLP, 125 SUMMER STREET, BOSTON, MA, 02110-1618, US

(21) Appl. No.: 11/207,643

(22) Filed: Aug. 19, 2005

Related U.S. Application Data
(60) Provisional application No. 60/606,583, filed on Sep. 2, 2004.

(51) Int. Cl. A61K 8/22; A61K 8/20

(52) U.S. Cl. 424/53

(57) ABSTRACT

A dental mouthwash product, and method of use, comprising a water-soluble chlorite compound in a first solution, a hydrogen peroxide and a physiologically acceptable readily water-soluble oxidizing agent in a second solution, or optionally the oxidizing agent separate from the second solution as a powder, tablet, gel capsule, or liquid, wherein said oxidizing agent oxidizes chlorite to ClO₂ within seconds, wherein the three ingredients are mixed to provide the mouthwash product. The mouthwash product may be used for any one or more of the following: as an antimicrobial agent for treating dental disease such as dental caries, plaque, gum disease, and/or bad breath, for strengthening gum and ligament attachments to the teeth, or for whitening teeth.

### CROSS REFERENCE TO RELATED APPLICATIONS

The present application claims priority from provisional application U.S. Application Ser. No. 60/606,583 filed Sep. 2, 2004, hereby incorporated herein by reference.

### TECHNICAL FIELD

The present invention relates to antimicrobial compositions suitable for prophylactic and therapeutic use, including the treatment and prevention of oral disease, and more particularly to systems capable of in situ generation of chlorine dioxide (ClO₂).

### BACKGROUND

The use of antimicrobial agents to treat and reduce oral and dental disease is well documented in the professional literature. Among the most efficacious such agents is ClO₂, a strong oxidizing agent. ClO₂ is well documented as a bactericidal, bacteriostatic, fungicidal, fungistatic, viricidal, and viralstatic agent. It is approved by the EPA under Registration Number 9048-3 for both water purification and food preparation and preservation because of this antimicrobial activity.

ClO₂ is also effective in treating malodor. It achieves this efficacy by two mechanisms of action. First, ClO₂ oxidizes the sulfide bonds of volatile and odoriferous sulfur compounds (specifically hydrogen sulfide and di-methyl mercaptan bonds) that are metabolic byproducts released by certain anaerobic bacteria documented to reside in the oral cavity; and second, its antimicrobial activity lowers the number of such microorganisms that release these volatile sulfur compounds.

However, because of its reactivity, ClO₂ is unstable in an aqueous solution and, as such, cannot be stored at room temperature. Furthermore, since ClO₂ is a gas, it cannot be stored in liquid form at room temperature. Thus, various references to “stabilized” ClO₂ do not refer to gaseous ClO₂, but rather to various chlorous acid-liberating compounds. Unfortunately, chlorous acid, even when buffered, will demineralize tooth enamel and lead to even more significant oral health problems.

One such chlorous acid-liberating compound used is sodium chlorite (NaClO₂). References to the use of NaClO₂ to generate chlorous acid can be found in the following papers: Chepek C W, Reed O K, Ratcliff P A, Reduction of Bleeding On Probing With Oral Care Products, Compendium 1995, 16(2): 188-196; Bolin V, Ratcliff P A, Germicidal Effect Of Providone Iodide and ClO₂ On Dental Pathogens. J. Dent Res. 1987, 373. IADR Abstracts; Grootveld M, Silwood C, Lynch E., Ability of Oral Heathcare Products to Alleviate Odour. J Dent. Res. 1997; 289:50. IADR Abstracts.

Compositions for treating oral malodor that employ chlorine-containing compounds are disclosed in U.S. Pat. No. 5,772,986 to Richter; U.S. Pat. No. 5,738,840 to Kross; U.S. Pat. No. 4,552,679 to Schubel, and U.S. Pat. No. 4,808,389 to Ratcliff. These references disclose various vehicles for introducing the compositions to the oral cavity, including liquid rinses, toothpastes (either with or without suds), lozenges, and sprays, as disclosed in U.S. Pat. No. 4,837,009 to Ratcliff. The chemical mechanisms for producing compositions containing chlorous acid are varied. Some references, such as Ratcliff '215, describe the generation of chlorous acid at controlled pH levels using phosphate buffers. U.S. Pat. No. 4,891,216 to Kross and U.S. Pat. No. 4,902,498 to Agricola et al. disclose a two part system that generates chlorous acid by mixing a metal chlorite or other chlorous acid-liberating compound with a protic acid at acidic pH levels. U.S. Pat. No. 5,667,817 to Kross discloses a two-stage system that requires the use of lactic acid and that results in a composition having a very disagreeable taste, making it unsuitable for use in oral healthcare. As a consequence, this product is not commercially available. However, even those products that are commercially available have significant drawbacks due to their complex chemistries, poor shelf life, poor taste, and poor efficacy.

Because chlorous acid will form ClO₂ in aqueous media, there will be some ClO₂ generated whenever chlorous acid contacts water. U.S. Pat. No. 6,280,775 to Sasson et al. discloses a two-stage formulation that generates therapeutically effective levels of ClO₂ upon admixture of a solution of H₂O₂ and an oxidant, preferably sodium persulfate with a solution of sodium chlorite. However, although the product disclosed in the '775 patent is able to provide therapeutic levels of ClO₂ in the presence of H₂O₂, the oxidant in the '775 patent is sodium persulfate which does not provide therapeutic levels of ClO₂ within seconds, and is not stable in the presence of hydrogen peroxide. In the presence of H₂O₂, sodium persulfate degrades very quickly, making it impossible to store the sodium persulfate and H₂O₂ in the same solutionIn addition, although the '775 composition does generate therapeutic levels of ClO₂ when the two solutions are mixed, those ClO₂ levels are not obtained for at least 2 minutes after mixing and at lower concentrations it takes even longer, which is not ideal for use as an oral rinse. And finally, sodium persulfate is a strong oxidizing agent that is regularly employed to adjust the pH of swimming pools in a process known as pool shock. It is also used to dye hair. Therefore, sodium persulfate is not an ideal physiologically acceptable oxidant for oral use.

Pending PCT Application “Antimicrobial Oral Composition and Method of Use” to Sasson et al., filed Feb. 23, 2004, is a continuation of PCT Application No. WO/0143732 filed Aug. 24, 2001, and discloses a two-stage system capable of in situ generation of chlorine dioxide in the presence of H₂O₂ and a physiologically acceptable oxidant having a reduction potential greater than 0.954 volts, preferably sodium persulfate. The continuation application discloses a number of oxidants which have the required reduction potential, including “salts of persulfate, iodate, bromate, permanganate, hypochlorite and the like”, as well as “organic peroxides” and “organic peracids” and “reagents which can be used to generate an oxidant in situ.” However, of the numerous oxidizing agents with the requisite reduction potential disclosed, whether generally or specifically, none successfully generated therapeutically effective levels of ClO₂ in situ, with the exception of the salts of persulfate—namely, sodium persulfate. The only effective composition in the pending PCT continuation application thus has the same deficiencies of that of the '775 patent—poor solubility and poor stability of the sodium persulfate oxidizing agent in aqueous H₂O₂ solutions, slow generation of ClO₂ levels, and controversial physiological acceptability of the sodium persulfate oxidizing agent.

Other known products that employ a single-stage system must control the spontaneous reaction that occurs between the metal chlorite and protic acid to form chlorous acid. For this purpose, various buffers must be used to regulate the system's pH below the pKa of chlorous acid, resulting in a relatively steady-state generation of chlorous acid. But, for these products to have any useful shelf-life, it is necessary that their steady-state ClO₂ levels be fairly low. Furthermore, because the reaction is unidirectional, not only is the product's shelf-life determined by the amount of metal chlorite initially present in the system and its pH, but the end-user is unable to determine how much chlorous acid is present at any given time, as the amount of chlorous acid in the system decays over time.

Commercially available, non-chlorous acid-containing products, such as Mentadent® (active ingredients: baking soda and H₂O₂) and Listerine® (active ingredients: thymol, eucalyptol, and methyl salicilate) oral rinses achieve plaque inhibition rates of only 15% and 30%, respectively. These levels are well below the therapeutic and prophylactic benchmark of about 50% plaque inhibition achieved by Peridex® oral rinse (active ingredient: chlorhexidine gluconate), which is available only by prescription. However, even though Peridex® is the most-effective, commercially available plaque inhibitor, it has serious drawbacks that limit its applicability. Most significant among these drawbacks is severe staining to hard oral tissues observed even with brief use. In addition to being unsightly, this black staining actually creates an environment for future plaque buildup, necessitating additional follow-up office visits to be removed by abrasion of the tooth surface, which, in turn, increases the teeth's susceptibility to caries.

There is a need for an oral, non-prescription multi-functional composition that rapidly generates therapeutic levels of ClO₂ within seconds in situ in the presence of a physiologically acceptable, water-soluble oxidant that is stable in the presence of 1.5% aqueous H₂O₂ solutions. Upon administration, the composition may achieve one or all of the following: plaque inhibition rates comparable to compositions currently available only by prescription, inhibition of gingival inflammation and periodontal inflammation, inhibition and a decrease in subgingival infection, reduction of dental caries, control of oral malodor, strengthening of gum structure, and fortification of ligaments and aid in ligament reattachment to teeth, and whitening of teeth. In addition, the composition should not stain the teeth, should not provide an environment for future plaque buildup, or should not make the teeth more susceptible to caries.

### SUMMARY OF THE INVENTION

The present invention is directed to a composition, and methods of treating or reducing the risk of a microbial infection using the composition made by mixing a solution of a water soluble metal chlorite with a solution of hydrogen peroxide and an oxidant such as ascorbic acid or tannic acid. The composition is especially useful as an oral rinse for treating or reducing the risk those microbial infections associated with dental disease, such as gingivitis, dental caries and oral malodor. The method additionally may whiten teeth, as an added cosmetic benefit. The system is capable of generating sufficient ClO₂ within seconds, so that, when used in the oral cavity, the composition may effect one or more of the following: inhibition of plaque formation, inhibition of gingival inflammation and/or periodontal inflammation, inhibition and/or decrease of sub-gingival infection, reduction of dental caries, control of oral malodor, strengthening of gum structure and ligament fortification/reattachment, and teeth whitening.

In one embodiment, there is provided a method of treating or reducing the risk of microbial infection comprising the steps of providing a first solution comprising a water-soluble chlorite compound, said chlorite compound present at a concentration in the range of about 0.1 to 0.5% by weight; providing a second solution comprising a physiologically acceptable readily water-soluble oxidizing agent and hydrogen peroxide, wherein said oxidizing agent oxidizes chlorite to ClO₂ within seconds and is present at a concentration in the range of about 0.01 to 33% by weight, and wherein said hydrogen peroxide is present at a concentration in the range of about 0.01 to 1.5% by weight; mixing the first solution and the second solution to provide an antimicrobial composition; and applying the composition to the locus of the microbial infection.

The oxidizing agent may be any of ascorbic acid, erythorbic acid, tannic acid, folic acid, retinoic acid, sorbic acid, adipic acid, glutathione, t-butylhydroperoxide, and 2,4-hexadienal or any other physiologically acceptable oxidizing agent that is stable in 1.5% H₂O₂ and generates ClO₂ from ClO₂⁻ within seconds, or mixtures thereof.

Another embodiment provides a method of treating or reducing the risk of microbial infection comprising the steps of providing a first solution comprising a water-soluble chlorite compound, said chlorite compound present at a concentration in the range of about 0.1 to 0.5% by weight; providing a second solution comprising ascorbic acid and hydrogen peroxide, wherein said ascorbic acid oxidizes chlorite to ClO₂ within seconds and is present at a concentration in the range of about 0.01 to 33% by weight, particularly about 0.1 to 30%, more particularly about 0.5 to 15%, and still more particularly about 1 to 6%, and wherein said hydrogen peroxide is present at a concentration in the range of about 0.01 to 1.5% by weight; mixing the first solution and the second solution to provide an antimicrobial composition; and applying the composition to the locus of the microbial infection.

Still another embodiment provides a method of treating or reducing the risk of dental disease comprising the steps of providing a water-soluble chlorite compound in a first solution; providing hydrogen peroxide and an oxidizing agent that is stable in the presence of 1.5% hydrogen peroxide and that oxidizes chlorite to ClO₂ within seconds, the hydrogen peroxide present in a second solution optionally containing the oxidizing agent; mixing the first solution and the second solution and oxidizing agent to produce a mixed composition; and applying the mixed composition as a mouthwash on a regular basis.

Alternative embodiments provide a method of treating or reducing the risk of microbial infection, or provide a method of treating or reducing the risk of dental disease wherein the oxidizing agent may be any of ascorbic acid, erythorbic acid, tannic acid, folic acid, retinoic acid, sorbic acid, adipic acid, glutathione, t-butylhydroperoxide, and 2,4-hexadienal or any other physiologically acceptable oxidizing agent that is stable in 1.5% H₂O₂ and generates ClO₂ from ClO₂⁻ within seconds, or mixtures thereof.

An alternative embodiment for treating or reducing the risk of dental disease comprises the steps of providing a water-soluble chlorite compound in a first solution; providing hydrogen peroxide and ascorbic acid, the hydrogen peroxide present in a second solution optionally containing the ascorbic acid; mixing the first solution and the second solution and ascorbic acid to produce a mixed composition; and applying the mixed composition as a mouthwash on a regular basis.

Particular embodiments for treating or reducing the risk of dental disease may also have one or more of the following features: the water-soluble chlorite compound in the first solution may be present at a concentration in the range of about 0.25 to 0.5% by weight; the water-soluble chlorite compound may be NaClO₂; the oxidizing agent may be present in the second solution at a concentration in the range of about 0.01 to 33% by weight; the oxidizing agent may be added immediately after mixing the first and second solutions in the form of a powder, tablet, gel capsule, or liquid; the hydrogen peroxide in the second solution may be present at a concentration in the range of about 0.01 to 1.5% by weight; and the antimicrobial composition may have a pH in the range of 1-8 after mixing.

In still more particular embodiments, the water-soluble chlorite compound in the first solution may be present at a concentration in the range of about 0.25 to 0.5% by weight; the water-soluble chlorite compound may be NaClO₂; the oxidizing agent may be ascorbic acid and be present in the second solution at a concentration in the range of about 0.1 to 30%, more particularly about 0.5 to 15%, and still more particularly about 1 to 6%, by weight; the hydrogen peroxide in the second solution may be present at a concentration in the range of about 0.01 to 1.5% by weight; and the mixed composition may have a pH in the range of 2-8 after mixing.

In still more particular embodiments, the oxidizing agent may be ascorbic acid and be added immediately after mixing the first and second solutions in the form of a powder, tablet, gel capsule, or liquid to achieve a concentration in the range of about 0.01 to 33% by weight, particularly about 0.1 to 30%, more particularly about 0.5 to 15%, and still more particularly about 1 to 6%; and the mixed composition may have a pH in the range of 2-8 after mixing.

In addition, other particular embodiments may provide a final composition that further comprises one or more of the following ingredients: fluoride, a colorant, a sweetener, a flavoring and a surfactant.

In yet another embodiment, there is provided a method of treating or reducing the risk of a microbial infection comprising the step of applying to the locus of the microbial infection, an antimicrobial composition comprising about 1-100 ppm chlorine dioxide, about 0.01-33% by weight of a physiologically acceptable readily water-soluble oxidizing agent that is stable in 1.5% H₂O₂ and oxidizes chlorite to ClO₂ within seconds, about 0.15-1.5% by weight of hydrogen peroxide, and about 0.05 to 0.5% chlorite. In a more particular embodiment, and the antimicrobial composition may comprise about 2 to 20 ppm chlorine dioxide generated from NaClO₂, and about 0.01 to 33%, particularly about 0.1 to 30%, more particularly about 0.5 to 15%, and still more particularly about 1 to 6%, by weight, ascorbic acid added just prior to treatment. In other embodiments, the antimicrobial composition comprises about 2 to 20 ppm chlorine dioxide, about 1.5 to 15% ascorbic acid, about 0.15 to 0.75% hydrogen peroxide, and about 0.05 to 0.5% NaClO₂. In still other embodiments, the antimicrobial composition may be an oral rinse for treating dental disease such as gingival plaque formation, dental caries or oral malodor, and may also be an oral rinse for whitening. In particular embodiments, the quantities of chlorite, oxidizing agent and hydrogen peroxide are based on the weight of the antimicrobial composition.

In other embodiments, there is provided a dental mouthwash product comprising a water-soluble chlorite compound, a physiologically acceptable readily water soluble oxidizing agent and hydrogen peroxide, wherein the oxidizing agent is stable in 1.5% H₂O₂ and oxidizes chlorite to ClO₂ within seconds and wherein the oxidizing agent may be any of ascorbic acid, erythorbic acid, tannic acid, folic acid, retinoic acid, sorbic acid, adipic acid, glutathione, t-butylhydroperoxide, and 2,4-hexadienal or any other physiologically acceptable oxidizing agent that is stable in 1.5% H₂O₂ and oxidizes chlorite to ClO₂ within seconds, and mixtures thereof. More particularly, the dental mouthwash product may comprise a dispensing container which houses a first compartment with an outlet end containing a first liquid component comprising the chlorite compound; and a second compartment with an outlet end containing a second liquid component comprising the H₂O₂ and optionally the oxidizing agent, a closure mechanism for closing the compartments over the outlet ends, and a closure means for allowing the first and second liquid components to be simultaneously dispensed. Alternatively, the oxidizing agent is added to the mouthwash product in the form of a powder, tablet, gel capsule, or liquid immediately after simultaneously dispensing the first and second liquid components.

In particular embodiments, the chlorite compound is present in the mouthwash product at a concentration in the range of about 0.1 to 0.5% by weight, the H₂O₂ is present at a concentration of about 0.01 to 1.5% by weight, and the oxidizing agent is present at a concentration of about 0.01 to 33% by weight.

A more particular embodiment provides an aqueous solution comprising about 0.05 to 0.5% ClO₂⁻, about 0.3 to about 1.5% H₂O₂ by weight, about 0.1 to 30%, more particularly about 0.5 to 15%, and still more particularly about 1 to 6%, by weight, of a physiologically acceptable oxidizing agent that generates ClO₂ from ClO₂⁻ within seconds, and which is stable in the present of 1.5% H₂O₂, and about 1 to 100 ppm ClO₂, wherein the oxidizing agent may be any of ascorbic acid, erythorbic acid, tannic acid, folic acid, retinoic acid, sorbic acid, adipic acid, glutathione, t-butylhydroperoxide, and 2,4-hexadienal, or any other physiologically acceptable oxidizing agent that generates ClO₂ from ClO₂⁻ within seconds, and which is stable in the present of 1.5% H₂O₂, or mixtures thereof. Alternatively, the oxidizing agent may be a salt of ascorbate.

Still other embodiments provide an antimicrobial mouthwash comprising a water-soluble chlorite compound, hydrogen peroxide, and an oxidizing agent, wherein the oxidizing agent is stable in the presence of 1.5% H₂O₂, oxidizes chlorite to ClO₂ within seconds, and may be any of ascorbic acid, erythorbic acid, tannic acid, any other physiologically acceptable oxidizing agent that is stable in the presence of 1.5% H₂O₂ and oxidizes chlorite to ClO₂ within seconds, or mixtures thereof.

Alternatively, the water-soluble chlorite compound is present in a first solution at a concentration in the range of about 0.1 to 0.5% by weight, the H₂O₂ is present in a second solution at a concentration of about 1.5% or less, by weight, and the oxidizing agent is optionally present in the second solution at a concentration of about 0.01 to 33% by weight, particularly about 0.1 to 30%, more particularly about 0.5 to 15%, and still more particularly about 1 to 6%, or added as a tablet immediately after mixing the first and second solutions.

In particular embodiments, the oxidizing agent is present in the second solution, and appropriate volumes of the first and second solutions to generate chlorine dioxide are mixed prior to use as a mouthwash. In more particular embodiments, the oxidizing agent is added in the form of a powder, tablet, gel capsule, or liquid immediately after mixing appropriate volumes of the first and second solutions, for example, the oxidizing agent is added as an effervescent tablet.

Another embodiment provides a method of whitening teeth comprising the steps of providing a water-soluble chlorite compound in a first solution, providing hydrogen peroxide and an oxidizing agent that is stable in the presence of 1.5% hydrogen peroxide and that oxidizes chlorite to ClO₂ within seconds, the hydrogen peroxide present in a second solution optionally containing the oxidizing agent, mixing the first solution and the second solution and oxidizing agent to produce a mixed composition, and applying the mixed composition as a mouthwash on a regular basis. In more particular embodiments, the oxidizing agent is present in the second solution and is any of ascorbic acid, erythorbic acid, tannic acid, folic acid, retinoic acid, sorbic acid, adipic acid, glutathione, t-butylhydroperoxide, and 2,4-hexadienal or any other physiologically acceptable oxidizing agent that is stable in 1.5% H₂O₂ and oxidizes chlorite to ClO₂ within seconds, and mixtures thereof

A still more particular embodiment provides a method of whitening teeth wherein the oxidizing agent is added in the form of a powder, tablet, gel capsule or liquid immediately after mixing the first and second solutions, and is any of ascorbic acid, erythorbic acid, tannic acid, folic acid, retinoic acid, sorbic acid, adipic acid, glutathione, t-butylhydroperoxide, and 2,4-hexadienal or any other physiologically acceptable oxidizing agent that is stable in 1.5% H₂O₂ and oxidizes chlorite to ClO₂ within seconds, and mixtures thereof. More particularly, the chlorite may be present in the first solution at a concentration in the range of about 0.05 to 0.5%, the hydrogen peroxide may be present in the second solution at a concentration of about 0.1 to 1.5%, by weight, and the oxidizing agent may be present in the mixed composition at a concentration of between about 0.01 to 33% by weight.

Another particular embodiment provides a method of strengthening gum and ligament attachments to teeth comprising the steps of providing a water-soluble chlorite compound in a first solution, providing hydrogen peroxide and ascorbic acid, the hydrogen peroxide present in a second solution optionally containing the ascorbic acid, mixing the first solution and the second solution and ascorbic acid to produce a mixed composition, and applying the mixed composition as a mouthwash on a regular basis.

In more particular embodiments for strengthening gum and ligament attachments to teach, one or more of the following features is also present: the water-soluble chlorite compound in the first solution is present at a concentration in the range of about 0.25 to 0.5% by weight, the water-soluble chlorite compound is NaClO₂, the ascorbic acid is present in the second at a concentration in the range of about 0.01 to 33% by weight; the ascorbic acid is added immediately after mixing the first and second solutions in the form of a powder, tablet, gel capsule, or liquid, the hydrogen peroxide in the second solution is present at a concentration in the range of about 0.01 to 1.5% by weight, and the antimicrobial composition has a pH in the range of 1-8 after mixing. Alternatively, the water-soluble chlorite compound in the first solution may be present at a concentration in the range of about 0.25 to 0.5% by weight, the water-soluble chlorite compound may be NaClO₂, the ascorbic acid may be present in the second solution at a concentration in the range of about 0.1 to 30%, more particularly about 0.5 to 15%, and still more particularly about 1 to 6%, by weight, the hydrogen peroxide in the second solution may be present at a concentration in the range of about 0.01 to 1.5% by weight, and the mixed composition may have a pH in the range of 2-8 after mixing. More particularly, the ascorbic acid may be added immediately after mixing the first and second solutions in the form of a powder, tablet, gel capsule, or liquid to achieve a concentration in the range of about 0.01 to 33%, particularly about 0.1 to 30%, more particularly about 0.5 to 15%, and still more particularly about 1 to 6%, by weight.

In yet another embodiment there is provided a method of preventing dental caries comprising the steps of providing a water-soluble chlorite compound in a first solution, providing hydrogen peroxide and ascorbic acid, the hydrogen peroxide present in a second solution optionally containing the ascorbic acid, mixing the first solution and the second solution and ascorbic acid to produce a mixed composition, and applying the mixed composition as a mouthwash on a regular basis.

More particularly, the method of preventing dental caries may further comprise one or more of the following features: the water-soluble chlorite compound in the first solution may be present at a concentration in the range of about 0.25 to 0.5% by weight, the water-soluble chlorite compound may be NaClO₂, the ascorbic acid may be present in the second at a concentration in the range of about 0.01 to 33% by weight, the ascorbic acid may be added immediately after mixing the first and second solutions in the form of a powder, tablet, gel capsule, or liquid, the hydrogen peroxide in the second solution may be present at a concentration in the range of about 0.01 to 1.5% by weight, and the antimicrobial composition may have a pH in the range of 1-8 after mixing. In an alternative embodiment, the ascorbic acid is present in the second solution at a concentration in the range of about 0.1 to 30%, more particularly about 0.5 to 15%, and still more particularly about 1 to 6%, by weight. In yet another alternative embodiment, the ascorbic acid is added immediately after mixing the first and second solutions in the form of a powder, tablet, gel capsule, or liquid to achieve a concentration in the range of about 0.01 to 33% by weight.

The specific examples and embodiments herein disclosed are illustrative only, and not intended to be limiting in any way. Modifications and substitutions that fall within the spirit and scope of the presently claimed invention are also considered be covered by the disclosure.

### DETAILED DESCRIPTION OF SPECIFIC EMBODIMENTS

Definitions. As used in this description and the accompanying claims, the following terms shall have the meanings indicated, unless the context otherwise requires:

The term “hydrogen peroxide” as used herein includes hydrogen peroxide itself as well as any peroxide generator such as urea peroxide, zinc peroxide, calcium peroxide, sodium percarbonate and the like.

The term “readily water-soluble” as used herein means a compound that dissolves instantly, or with minimal stirring, at concentrations from 1% to 6% or greater (w/v), in solutions having pH values from about 5 to about 8.

The term “at or below a pH of about 7” means, in the context of this application, pH values from about 6 to about 8, plus or minus a half a pH unit.

The term “oxidizing agent stable in the present of 1.5% H₂O₂” means, in the context of this application an oxidizing agent that shows no depreciable degradation in the presence of 1.5% H₂O₂ for 5 days or longer, particularly for a month or longer, and more particularly for up to a year or indefinitely.

The term “applying . . . as a mouthwash on a regular basis” means, in the context of this application, applying the composition, solution, or liquid to the mouth, without swallowing for approximately 5 to 60 seconds, followed by spitting, preferably at least once a month, more preferably at least once a week, and more preferably more than once per week, for example, daily, or several times per day, depending on the need and desired effect.

A preferred water soluble chlorite compound for the first solution is NaClO₂. However, other water soluble metal salts of chlorite, including other alkali metal salts and group (II) salts (e.g., calcium and magnesium salts) can also be used. The water soluble chlorite is present at a concentration in the range of about 0.1 to 0.5% by weight, preferably about 0.25 to 0.32%. This first solution is maintained at an alkaline pH above about 7, preferably around pH 8.

The oxidizing agent is required to be readily water soluble, and physiologically acceptable, i.e., suitable for administration to humans. The oxidizing agent is also required to be suitable for oxidizing the chlorite to ClO₂. Surprisingly, of a large number of physiologically acceptable oxidizing agents tested, only ascorbic acid and tannic acid successfully generated therapeutically effective levels of ClO₂ in situ under a variety of pH ranges and concentrations, whether within seconds or a substantially longer period of time. Examples of other physiologically acceptable oxidizing agents which do not work and in fact generated no ClO₂ under the conditions tested include adipic acid, folic acid, retinoic acid, sorbic acid, folic acid, glutathione, 2,4-hexadienal and t-butylhydroperoxide. That ascorbic acid and tannic acid generate therapeutic levels of ClO₂ within seconds, even at low concentrations, is even more surprising given that all the above-discussed failure oxidants have a reduction potential greater than 0.954 and so should have worked (according to the disclosure and claims of PCT Application No. WO/0143732) but in fact did not work. This exemplifies the unpredictable nature of identifying acceptable oxidants and completely refutes prior claims that any oxidant with a given reduction potential can be used.

Therefore, oxidants of the presently claimed invention particularly include ascorbic acid (solubility of approximately 33% in water at standard temperature and pressure), erythorbic acid and tannic acid (solubility of 1 g/0.35 mL water, standard temperature and pressure), each being a preferred oxidant, but any non-toxic oxidant which, when mixed with H₂O₂ and a metal chlorite, generates ClO₂ in situ falls within the scope of the invention.

The second solution comprises hydrogen peroxide (H₂O₂), optionally an oxidizing agent (such as ascorbic acid) and is maintained at a pH in the range of about 1 to 8. If the oxidizing agent is not present in the second solution, it is added as a tablet immediately after mixing the first and second solutions. In the mixed solution produced, he oxidizing agent is present at a concentration in the range of about 0.01 to 33%, preferably in the range of about 3 to 20%. If the ascorbic acid is present in the first solution with the H₂O₂ and not added as a tablet, powder, or gel, after mixing the first and second solutions, then the resulting mixed solution will have a final concentration of about 16% because of the limit of solubility by weight of ascorbic acid in water (about 33% by weight). The hydrogen peroxide is present at a concentration in the range of 0.01 to 1.5% by weight, preferably in the range of about 0.3 to 1.5%.

Oxidation reactions involving H₂O₂ solutions are generally complicated and difficult to elucidate—a situation made more complicated by the presence of an oxidizing agent that is also an acid, such as ascorbic acid. And, while this invention is not intended to be limited by any particular theory or mechanism of action, it is believed that the therapeutic effectiveness of the present compositions is due at least in part to the formation of chlorine dioxide (ClO₂). When the first and second solutions are mixed and suitable oxidizing agent is present, ClO₂ is formed by the oxidation of the water-soluble chlorite compound as described by the reactions shown below. Chlorine dioxide is a known antimicrobial agent. Hydrogen peroxide is also believed to be responsible in part for the therapeutic effectiveness of the present compositions, even though it is known that in the presence of H₂O₂, ClO₂ is reduced back to ClO₂⁻ and hydrogen peroxide is consumed.

Applicants have found that when there is an adequate amount of oxidizing agent, as described herein, relatively small amounts of water soluble metal chlorite and hydrogen peroxide may be used together to provide an effective oral rinse.

The amounts of water soluble metal chlorite compound, hydrogen peroxide and oxidizing agent ingredients described above are based on mixing the first and second solutions in approximately appropiate volumes, wherein the oxidizing agent is optionally present in the second solution, or added immediately after mixing the first and second solutions, for example, in the form of a powder, tablet, gel capsule, or liquid. These volumes may be varied to adjust for variations in the concentration of the ingredients in the first and second solutions. In one particular embodiment, the second solution comprises hydrogen peroxide and oxidant. In another particular embodiment the second solution comprises hydrogen peroxide with no oxidant, and the oxidant, particularly ascorbic acid in tablet form, is added to the make the active oral composition immediately following admixture of the first and second solutions.

Accordingly, another embodiment of the invention relates to a method for treating or reducing the risk of a microbial infection comprising the step of applying an antimicrobial composition having a pH at or below about 7 to the locus or surface of the microbial infection, wherein said composition is prepared by mixing a first solution with a second solution, the first solution comprising a water soluble metal chlorite and the second solution comprising a suitable oxidizing agent, as described above (e.g., ascorbic acid), and hydrogen peroxide. The resulting mixture comprises about 0.05 to 0.25% of the water soluble metal chlorite, about 0.5 to 6% or higher of the oxidizing agent (e.g., ascorbic acid) and about 0.15 to 0.75% hydrogen peroxide, wherein all quantities are based on the weight of the antimicrobial composition.

The antimicrobial composition obtained after mixing the solutions will have ClO₂ at a concentration in the range of about 1 to 100 ppm within 30 seconds of mixing, preferably in the range of about 2 to 50 ppm within 10 seconds of mixing; hydrogen peroxide in the range of about 0.15 to 0.75%, preferably in the range of about 0.15 to 0.4%; oxidizing agent in the range of about 0.01 to 33% by weight, particularly about 0.1 to 30%, more particularly about 0.5 to 15%, and still more particularly about 1 to 6%, especially any concentration of oxidant that generates 200 ppm or less within a 2 minute period of time, preferably about 1.5 to about 15%; and ClO₂⁻ in the range of about 0.05 to 0.5%. Accordingly, another embodiment of this invention relates to a method for treating or reducing the risk of microbial infection comprising the step of applying to the locus of the microbial infection an antimicrobial composition comprising (a) ClO₂ at a concentration in the range of about 1 to 100 ppm, preferably in the range of about 2 to 50 ppm; (b) hydrogen peroxide in the range of about 0.15 to 0.75% by weight, preferably in the range of about 0.15 to 0.4%; (c) oxidizing agent in the range of about 0.01 to 33%, particularly about 0.1 to 30%, more particularly about 1.5 to 15%, and still more particularly about 1 to 6%; and (d) ClO₂⁻ in the range of about 0.05 to 0.5% by weight, wherein the pH of the composition is at or below about 7. Another embodiment of the present invention is the aforementioned composition formed from mixing the two solutions.

For adjusting the pH, any suitable buffer may optionally be used such as a bicarbonate buffer, a citrate buffer or a phosphate buffer. When used, the buffer will typically be present at a concentration in the range of about 0.1 to 1.0% by weight. Bicarbonate is a preferred buffer system. Any suitable food-grade acid or base may be used to prepare the buffer system or to otherwise adjust the pH. Preferred acids are phosphoric acid and citric acid, optionally supplemented by tannic acid, and a preferred base is sodium bicarbonate.

The composition may contain optional ingredients to improve taste, appearance or mouth feel in order to enhance its appeal to the consumer. Such optional ingredients include colorants, sweeteners, flavorings and surfactants that are known ingredients in commercially available mouthwash. Examples of colorants include FDC Red 40, FDC Green 3, FDC Brown mixture, FDC Yellow 5, DC Red 19, DC Red 33, DC Yellow 10, and the like, which are typically present in about 0.01 to 0.2 weight percent. Examples of suitable sweeteners include glycerin and sugar alcohols like sorbitol or artificial sweeteners such as aspartame, saccharin or acesulfame. Sweetening agents are generally used at levels of from about 0.005% to about 2% by weight of composition. Examples of flavorants include oils of spearmint, peppermint, wintergreen, sassafras, clove, sage, eucalyptus, cinnamon, lemon, orange, and methyl salicylate. Flavorants are used in a quantity of about 0.1 percent by weight. A surfactant can be added as an optional ingredient in a quantity of about 0.2-2 weight percent, and preferably is selected from orally-compatible nonionic and anionic polymers which are commercially available for oral hygiene applications. Nonionic oral surfactants are illustrated by laurate esters of sorbitol consisting of the monoester condensed with about 15-25 moles of ethylene oxide, such as Tween 20 (ICI Americas). Another suitable type of oral surfactants are the polymers of polyoxyethylene and polyoxypropylene, such as Pluronic F-108 (BASF-Wyandotte). Anionic oral surfactants are illustrated by alkyl sulfonates and sulfates, such as sodium lauryl sulfate or a sulfonated monoglyceride of a C 10-C 18 fatty acid. The present composition may also contain deodorizing agents, anti-foam agents, ethanol or other alcohols, as well as other conventional ingredients. In addition, the present composition may include fluoride at a concentration in the range of about 0.01% to 1.25%.

It is preferred that the present composition be prepared fresh from the two solutions and used at or near the time of preparation. The composition is best used between about 1 to 5 minutes after it is prepared. However, if the composition is left to sit for several hours, its effectiveness will begin to diminish. In the field of oral mouthwashes, suitable containers have been developed for maintaining separate solutions that are to be mixed at the time of use by the consumer. U.S. Pat. Nos. 5,252,312, 5,289,950 and 5,392,947, incorporated herein by reference, describe such dispensing containers for dental mouthwash. The containers have at least two discreet compartments so that the contents of the compartments may be dispensed simultaneously.

Accordingly, another embodiment of this invention is directed to a kit or dental mouthwash product comprising two components. The kit or dental mouthwash product can be used in therapy, for example, for treating and/or reducing the risk of microbial infections such as those associated with dental disease. The components can also be used in the manufacture of a medicament for use in treating or reducing the risk of said microbial infections. The kit or dental mouthwash product comprises a first liquid component comprising a water-soluble chlorite compound present at a concentration in the range of about 0.1 to 0.5% by weight, wherein the first liquid component has an alkaline pH; and a second liquid component comprising a second solution of an oxidizing agent, as described herein (preferably ascorbic acid) and hydrogen peroxide, wherein the oxidizing agent is present at a concentration in the range of about 0.01 to 33% by weight, the hydrogen peroxide is present at a concentration in the range of about 0.01 to 3% by weight, and the second component has a pH in the range of about 1 to 8.

Optionally, the kit or dental mouthwash product additionally comprises a dispensing container which houses a first compartment with an outlet end containing the first liquid component and a second compartment with an outlet end containing the second liquid component; a closure mechanism for closing the compartments over the outlet ends; and a closure means for allowing the first and second liquid components to be simultaneously dispensed.

Such containers are especially well-suited to simultaneously dispensing equal quantities of the two liquid components. Optionally, the outer walls of one or both of the compartments may be constructed of a translucent or clear material so that the liquid level within the container may be viewed.

The composition may be used as an oral rinse for treating dental disease, and/or for whitening teeth. Such disease includes gingivitis, dental caries and oral malodor. The amount of composition and the frequency of treatment may be varied depending on the type and severity of the disease and on the mode of application. The amount of composition used per treatment may vary from about 0.1 ml to 100 ml depending on the application. The lower amounts may be sufficient if the composition is to be applied directly, for example, by using a syringe or other means of direct application. For an oral rinse, the amounts typically vary from a few milliliters to about 100 mL per treatment, preferably from about 25 to 50 mL. The composition may be used as a mouthwash daily or multiple times during the day or in accordance with a treatment regimen that would be prescribed by one skilled in the art of dental care. For treating the dental diseases described herein, treatments will generally be made once to a few times per day, preferably twice per day.

The composition may also be used in dental appliance therapy, especially for treating extra-oral appliances such as removable partial dentures, full dentures, night guards, and orthodontic appliances. The appliance is treated by immersing it in the composition for a suitable period of time, usually about 10 to 15 minutes.

The pH of the resulting composition should be at or below about 7, but above the pKa of chlorous acid. It is known that ClO₂ is generated from NaClO₂ at pH values at or below about 7 in the absence of an oxidizing agent such as C₆H₈O₈. However, the generation is quite slow (on the order of days and months). In the presence of an oxidizing agent such as C₆H₈O₈ or tannic acid, under the conditions described above, therapeutically effective levels of ClO₂ begin to be generated in seconds, as shown below in Tables 1 and 2. Increasing the concentration of the oxidizing agent by weight will cause a more rapid generation of ClO₂. Tables 1A and 1B show that for a constant level of NaClO₂ the generation of ClO₂ increases with increasing levels of C₆H₈O₈ or tannic acid, respectively, as measured by a change in absorption at 360 nm.

TABLE 1A
Time-course of generation of ClO₂ (in seconds) after a 1:1 mixture of a NaClO₂
solution (0.25%) and a solution of C₆H₆O₈²⁻ (ranging from 1-6%) at pH = 6,
as measured by change in absorption at 360 nm (ΔA₃₆₀).

| | 1% | 2% | 3% | 4% | 5% | 6% |
|---|---|---|---|---|---|---|
| Time (s) | C₆H₆O₈²⁻ | C₆H₆O₈²⁻ | C₆H₆O₈²⁻ | C₆H₆O₈²⁻ | C₆H₆O₈²⁻ | C₆H₆O₈²⁻ |
| 10 | 0.065700 | 0.131468 | 0.197279 | 0.253301 | 0.336816 | 0.398394 |
| 20 | 0.091049 | 0.182592 | 0.274094 | 0.293990 | 0.462512 | 0.552951 |
| 30 | 0.107051 | 0.214380 | 0.322064 | 0.323223 | 0.538073 | 0.648060 |
| 40 | 0.124580 | 0.249206 | 0.374635 | 0.401855 | 0.627210 | 0.752433 |
| 50 | 0.141988 | 0.284278 | 0.426890 | 0.592883 | 0.713627 | 0.861771 |
| 60 | 0.154237 | 0.308966 | 0.463183 | 0.701213 | 0.777724 | 0.930061 |

TABLE 1B
Time-course of generation of ClO₂ (in seconds) after a 1:1 mixture of a NaClO₂
solution (0.25%) and a solution of tannic acid (T.A. - ranging from 1-6%)
at pH = 6, as measured by change in absorption at 360 nm (ΔA₃₆₀).

| Time (s) | 1% T.A. | 2% T.A. | 3% T.A. | 4% T.A. | 5% T.A. | 6% T.A. |
|---|---|---|---|---|---|---|
| 10 | 0.049285 | 0.100196 | 0.151265 | 0.202061 | 0.253532 | 0.301514 |
| 20 | 0.062938 | 0.132889 | 0.194910 | 0.260667 | 0.322099 | 0.381255 |
| 30 | 0.099178 | 0.202198 | 0.303601 | 0.405643 | 0.496425 | 0.597066 |
| 40 | 0.114933 | 0.233982 | 0.348199 | 0.460542 | 0.575160 | 0.697547 |
| 50 | 0.128531 | 0.261357 | 0.393821 | 0.520451 | 0.643029 | 0.778785 |
| 60 | 0.140002 | 0.288637 | 0.429665 | 0.565916 | 0.700927 | 0.840591 |

In addition, FIG. 1 shows the ΔA₃₆₀ values in Tables 1A and 1B plotted as a function of concentration. As can be seen, the ΔA₃₆₀ values increase linearly with increasing concentration.

As discussed above, although an oxidant, in this reaction H₂O₂ will reduce ClO₂ back into NaClO₂. In the above stoichiometry, ClO₂ is generated even in the presence of H₂O₂. While the apparent ClO₂ concentration decreases with increasing levels of H₂O₂, even at the highest levels of H₂O₂ (1.2%), there is sufficient ClO₂ to inhibit gingival plaque. For products requiring less H₂O₂, either higher levels of ClO₂ can be obtained or the levels of NaClO₂ and oxidizing agent may be adjusted, in a manner that would be apparent to one skilled in the art, to yield desired levels of ClO₂.

In the case where the application also calls for the use of NaHCO₃, such as in bicarbonate- and peroxide-containing products, the pH of the ascorbate/peroxide-containing component should be sufficiently acidic such that upon mixing with the NaClO₂/bicarbonate-containing component the resulting pH is at or less than about 7.

Table 2 below shows a formula for a two-component system in accordance with one embodiment of the present invention. The ranges given below represent the various conditions that could result in levels of ClO₂ that inhibit plaque. In general the ClO₂ levels will be dependent on the level of NaClO₂ in the base and the level of H₂O₂ and C₆H₈O₈ in the activator. The level of NaHCO₃ in the base will determine the level of buffer, for example phosphoric acid (H₃PO₄), that should be used in order to adjust the pH of the activator. Upon mixing the base and activator, the resulting pH should be at or below about 8. Increasing levels of NaHCO₃ in the base therefore will require increasing levels of buffer in the activator to achieve a final pH of about 8 or less when the two phases are mixed.

TABLE 2
Formulation for Two-Component System.

| | |
|---|---|
| Base | Activator |
| (Solution 1) | (Solution 2) |

| Component | Wt. % | Component | Wt. % |
|---|---|---|---|
| NaClO₂ | 0.1-0.5 | H₂O₂ | 0.3-1.5 |
| NaHCO₃ | up to 1.0 | C₆H₈O₈ | 1.0-10.0 |
| Surfactant | 0.01-1.0 | Surfactant | 0.01-1.0 |
| Flavor | 0.5-2.0 | Flavor | 0.5-2.0 |
| Ethanol | as needed | buffer | as needed |
| Water | balance | Water | balance |

The lower levels of NaClO₂ and C₆H₈O₈ can be used to generate the desired levels of ClO₂ if the H₂O₂ is eliminated from the formulation. The level of NaHCO₃ will not effect the generation of ClO₂ where no H₂O₂ is present. However, the pH of the resulting solution should be at or below about 8 if NaHCO₃ is used in the formula. It may be desirable in some applications to include bicarbonate.

### EXAMPLE 1

### Plaque Inhibition Study

A standardized Ramfjord protocol is followed for all subjects in the study. Between 50 to 100 adult subjects, between the ages of 18 and 65, are recruited for a two-cell, 48 hour plaque inhibition study against a water placebo control. The study is conducted in a double blind manner using the formulations described in Tables 3 and 4 below. The subjects receive 50 mL unit doses, two times per day over a 48 hour period. Neither examiner nor subject has knowledge of the test product identity.

TABLE 3
Example clinical formula for evaluation in a double-blind, two-cell,
48 hour plaque inhibition study against a water placebo control.

| | |
|---|---|
| Base | Activator |

| Component | Wt. % | Component | Wt. % |
|---|---|---|---|
| NaClO₂ | 0.3 | H₂O₂ | 0.75 |
| NaHCO₃ | 0.% | C₆H₈O₈ | 6.0 |
| Surfactant | 0.5 | Surfactant | 0.5 |
| Flavor | 0.5 | Flavor | 0.5 |
| Ethanol | 10.0 | H₃PO₄ | as needed |
| Water | balance | Water | balance |

TABLE 4
Example clinical formula for evaluation in a double-blind, two-cell,
48 hour plaque inhibition study against a water placebo control.

| | |
|---|---|
| Base | Activator |

| Component | Wt. % | Component | Wt. % |
|---|---|---|---|
| NaClO₂ | 0.32 | H₂O₂ | 1.5 |
| NaHCO₃ | 0.5 | Tannic Acid | 5.0 |
| Surfactant | 0.5 | Surfactant | 0.5 |
| Flavor | 0.5 | Flavor | 0.5 |
| Ethanol | 10.0 | H₃PO₄ | as needed |
| Water | balance | Water | balance |

On day one the panelists receive dental prophylaxis and are instructed to use the assigned rinse. No oral hygiene regimens, besides the rinsing with the test product or placebo, are allowed. At the end of the two-day treatment, panelists receive a plaque evaluation of the Ramfjord teeth as well as all other molars, excluding third molars. The plaque are evaluated for supragingival plaque using the Distal Mesial Plaque Index scoring method.

The mean values calculated from the double-blind study will be mean plaque scores for each tooth surface examined on the Ramfjord teeth. The greater the mean value, the greater the relative plaque accumulation. The data are expected to show that the ClO₂ group mean plaque scores are significantly lower than the water placebo group mean scores for all individual surfaces and for the total mouth. It is expected that the ClO₂ rinse is significantly more efficacious than water in the inhibition of dental plaque on the teeth, over a 48 hour period with four applications.

### EXAMPLE 2

Similar to the protocol described in Example 1, volunteer subjects will evaluate the composition for efficacy in whitening teeth. Two groups of between 50 to 100 adult subjects, between the ages of 18 and 65, are recruited for a two-cell, one-month or two-month tooth-whitening study against a water placebo control. Each study (one-month or two-month) is conducted in a double blind manner using the formulations described in Tables 4 and 5 above. The subjects receive 50 mL unit doses, two times per day over a period of one or two months or longer. Volunteers are requested to rinse for a full 30 seconds before discarding the rinse. Neither examiner nor subject has knowledge of the test product identity.

On day one the panelists receive dental prophylaxis and are instructed to use the assigned rinse. No oral hygiene regimens, besides the rinsing with the test product or placebo, are allowed. Spectrophotometry of the teeth are taken before the beginning of the study, and then every two weeks, for a comparative basis, using standardized spectrophotometric conditions. Whiteness is evaluated by analyzing the shades of whiteness of the teeth obtained from the spectrophotometric analyses, and comparing the change in shades of whiteness measured at the beginning, end, and at various mid-points throughout the study, as appropriate. In addition, participants in the study are asked to subjectively assess whiteness themselves each week, using a rating scale of 1 to 5 checks, as a indicator of whether patient-perceived whitening is occurring, as compared to spectrophotometrically measurable whiteness.

Alternatively, a protocol similar to that used in “Efficacy of a Fluoridated Hydrogen Peroxide-Based Mouth Rinse for the Treatment of Gingivitis: A Randomized Clinical Trial” may be used by modifying the solutions tested to include chlorite and an oxidizing agent for generating ClO₂ in situ (see Hasturk et al., J. Periodontol. 75:1, pp 57-65 (2004)), which is incorporated herein by reference in its entirety.

### EXAMPLE 3

### Gum Strengthening Studies

Compositions of the present application may also be used to strengthen gum and ligament connections to teeth, due to the beneficial effects of ascorbic acid (vitamin C) on collagen production. (See “Role of Ascorbic Acid in Periodontal Ligament Cell Differentiation” in J. Periodontol., 75:5, pp. 709-716 (2004), which is incorporated herein by reference in its entirety).

### EXAMPLE 4

### Dental Cary Inhibition Studies

Studies will be carried out similarly to those described in example 1 above, and evidence of reduction of dental caries will be evaluated according to evidence for reduction in plaque since it is well established that an increase in plaque build-up is directly correlated to an increase in dental caries.

### EXAMPLE 5

### Multi-Effect Studies

Alternatively, a single randomized, double blind, single center, placebo-controlled, parallel-group design study is carried out using 50 to 100 subjects per trial (or more, as required). Clinical measures will be taken to reflect the effects of gingival and periodontal health and tooth whiteness. Test results may record the following, as indicators of plaque inhibition, control of malodor, inhibition of microbial infection, gum strengthening, and teeth whitening: Eastman gingival bleeding index for gum strength and inhibition of microbial infection; modified Quigley and Hein plaque index for plaque inhibition; microbial analysis of gingival bacterial shifts using DNA testing for inhibition of microbial infections; treatment/prevention of halitosis using either a halimeter or gas chromatography; and teeth whitening using Vita dental porcelain shade guides or spectrophotometric recordings.

In addition to use within the oral cavity, it is contemplated that the present invention has applicability for use on the vaginal, anal, nasal, and ocular mucous membrane surfaces, topically, and in vitro, and, e.g., as a contact lens wash or dental apparatus wash.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating or reducing the risk of microbial infection comprising the steps of:
a) providing a first solution comprising a water-soluble chlorite compound, said chlorite compound present at a concentration in the range of about 0.1 to 0.5% by weight;
b) providing a second solution comprising a physiologically acceptable readily water-soluble oxidizing agent and hydrogen peroxide, wherein said oxidizing agent oxidizes chlorite to ClO₂ within seconds and is present at a concentration in the range of about 0.01 to 33% by weight, and wherein said hydrogen peroxide is present at a concentration in the range of about 0.01 to 1.5% by weight
c) mixing the first solution and the second solution to provide an antimicrobial composition; and
d) applying the composition of step (c) to the locus of the microbial infection.

2. The method of claim 1 wherein the oxidizing agent is any of ascorbic acid, erythorbic acid, tannic acid, folic acid, retinoic acid, sorbic acid, adipic acid, glutathione, t-butylhydroperoxide, and 2,4-hexadienal or any other physiologically acceptable oxidizing agent that is stable in 1.5% H₂O₂ and generates ClO₂ from ClO₂⁻ within seconds, or mixtures thereof.

3. A method of treating or reducing the risk of microbial infection comprising the steps of:
a) providing a first solution comprising a water-soluble chlorite compound, said chlorite compound present at a concentration in the range of about 0.1 to 0.5% by weight;
b) providing a second solution comprising ascorbic acid and hydrogen peroxide, wherein said ascorbic acid oxidizes chlorite to ClO₂ within seconds and is present at a concentration in the range of about 0.01 to 33% by weight, and wherein said hydrogen peroxide is present at a concentration in the range of about 0.01 to 1.5% by weight
c) mixing the first solution and the second solution to provide an antimicrobial composition; and
d) applying the composition of step (c) to the locus of the microbial infection.

4. A method of treating or reducing the risk of dental disease comprising the steps of:
a) providing a water-soluble chlorite compound in a first solution;
b) providing hydrogen peroxide and an oxidizing agent that is stable in the presence of 1.5% hydrogen peroxide and that oxidizes chlorite to ClO₂ within seconds, the hydrogen peroxide present in a second solution optionally containing the oxidizing agent;
c) mixing the first solution and the second solution and oxidizing agent to produce a mixed composition; and
d) applying the mixed composition of step (c) as a mouthwash on a regular basis.

5. The method of claim 4 wherein the oxidizing agent is any of ascorbic acid, erythorbic acid, tannic acid, folic acid, retinoic acid, sorbic acid, adipic acid, glutathione, t-butylhydroperoxide, and 2,4-hexadienal or any other physiologically acceptable oxidizing agent that is stable in 1.5% H₂O₂ and generates ClO₂ from ClO₂⁻ within seconds, or mixtures thereof.

6. A method of treating or reducing the risk of dental disease comprising the steps of:
a) providing a water-soluble chlorite compound in a first solution;
b) providing hydrogen peroxide and ascorbic acid, the hydrogen peroxide present in a second solution optionally containing the ascorbic acid;
c) mixing the first solution and the second solution and ascorbic acid to produce a mixed composition; and
d) applying the mixed composition of step (c) as a mouthwash on a regular basis.

7. The method of claim 4, said method having one or more of the following features:
the water-soluble chlorite compound in the first solution is present at a concentration in the range of about 0.25 to 0.5% by weight;
the water-soluble chlorite compound is NaClO₂;
the oxidizing agent is present in the second solution at a concentration in the range of about 0.01 to 33% by weight;
the oxidizing agent is added immediately after mixing the first and second solutions in the form of a powder, tablet, gel capsule, or liquid;
the hydrogen peroxide in the second solution is present at a concentration in the range of about 0.01 to 1.5% by weight; and
the antimicrobial composition has a pH in the range of 1-8 after mixing.

8. The method of claim 6 wherein:
the water-soluble chlorite compound in the first solution is present at a concentration in the range of about 0.25 to 0.5% by weight;
the water-soluble chlorite compound is NaClO₂;
the oxidizing agent is ascorbic acid and is present in the second solution at a concentration in the range of about 0.01 to 33% by weight; the hydrogen peroxide in the second solution is present at a concentration in the range of about 0.01 to 1.5% by weight; and
the mixed composition has a pH in the range of 2-8 after mixing.

9. The method of claim 6 wherein:
the water-soluble chlorite compound in the first solution is present at a concentration in the range of about 0.25 to 0.5% by weight;
the water-soluble chlorite compound is NaClO₂;
the oxidizing agent is ascorbic acid and is added immediately after mixing the first and second solutions in the form of a powder, tablet, gel capsule, or liquid to achieve a concentration in the range of about 0.01 to 33% by weight; and
the mixed composition has a pH in the range of 2-8 after mixing.

10. The method of claim 6 wherein the final composition further comprises one or more of the following ingredients: fluoride, a colorant, a sweetener, a flavoring and a surfactant.

11. A method of treating or reducing the risk of a microbial infection comprising the step of applying to the locus of the microbial infection, an antimicrobial composition comprising (a) about 1-100 ppm chlorine dioxide; (b) about 0.01-33% by weight of a physiologically acceptable readily water-soluble oxidizing agent that is stable in 1.5% H₂O₂, oxidizes chlorite to ClO₂ within seconds; (c) about 0.15-1.5% by weight of hydrogen peroxide; and (d) about 0.05 to 0.5% chlorite.

12. A method of treating or reducing the risk of a microbial infection comprising the step of applying to the locus of the microbial infection, an antimicrobial composition comprising (a) about 1-100 ppm chlorine dioxide; (b) about 0.01-33% by weight of ascorbic acid; (c) about 0.15-1.5% by weight of hydrogen peroxide; and (d) about 0.05 to 0.5% chlorite

13. The method of claim 11 wherein the antimicrobial composition comprises about 2 to 20 ppm chlorine dioxide and about 0.01 to 33% ascorbic acid added just prior to use; and the water soluble chlorite compound is NaClO₂.

14. The method of claim 12 wherein the antimicrobial composition comprises (a) about 2 to 20 ppm chlorine dioxide; (b) about 1.5 to 15% ascorbic acid; (c) about 0.15 to 0.75% hydrogen peroxide; and (d) about 0.05 to 0.5% NaClO₂.

15. The method of claim 11 wherein the antimicrobial composition is an oral rinse for treating dental disease and whitening teeth.

16. The method of claim 3 or 11 wherein the dental disease is selected from gingival plaque formation, dental caries or oral malodor.

17. A method for treating or reducing the risk of a microbial infection comprising the step of applying an antimicrobial composition to the locus or surface of the microbial infection, wherein said composition is prepared by mixing a first solution with a second solution, the first solution comprising a water soluble metal chlorite and the second solution comprising a physiologically acceptable readily water-soluble oxidizing agent and hydrogen peroxide, wherein the oxidizing agent is stable in 1.5% H₂O₂ and oxidizes chlorite to ClO₂ within seconds, and wherein the composition comprises about 0.05 to 0.25% of the water soluble metal chlorite, about 0.01 to 33% of the oxidizing agent and about 0.15 to 1.5% hydrogen peroxide, wherein the quantities of chlorite, oxidizing agent and hydrogen peroxide are based on the weight of the antimicrobial composition.

18. A method for treating or reducing the risk of a microbial infection comprising the step of applying an antimicrobial composition to the locus or surface of the microbial infection, wherein said composition is prepared by mixing a first solution with a second solution, the first solution comprising a water soluble metal chlorite and the second solution comprising ascorbic acid and hydrogen peroxide, wherein the ascorbic acid is stable in 1.5% H₂O₂ and oxidizes chlorite to ClO₂ within seconds, and wherein the composition comprises about 0.05 to 0.25% of the water soluble metal chlorite, about 0.01 to 33% of ascorbic acid and about 0.15 to 1.5% hydrogen peroxide, wherein the quantities of chlorite, oxidizing agent and hydrogen peroxide are based on the weight of the antimicrobial composition.

19. A dental mouthwash product comprising:
a water-soluble chlorite compound;
a physiologically acceptable readily water soluble oxidizing agent and hydrogen peroxide, wherein said oxidizing agent is stable in 1.5% H₂O₂ and oxidizes chlorite to ClO₂ within seconds.

20. The dental mouthwash product of claim 19 wherein the oxidizing agent is any of ascorbic acid, erythorbic acid, tannic acid, folic acid, retinoic acid, sorbic acid, adipic acid, glutathione, t-butylhydroperoxide, and 2,4-hexadienal or any other physiologically acceptable oxidizing agent that is stable in 1.5% H₂O₂ and oxidizes chlorite to ClO₂ within seconds, and mixtures thereof.

21. The dental mouthwash product of claim 19 further comprising:
1) a dispensing container which houses a first compartment with an outlet end containing a first liquid component comprising the chlorite compound; and a second compartment with an outlet end containing a second liquid component comprising the H₂O₂ and optionally the oxidizing agent;
2) a closure mechanism for closing the compartments over the outlet ends; and
3) a closure means for allowing the first and second liquid components to be simultaneously dispensed.

22. The dental mouthwash product of claim 19, wherein the oxidizing agent is added in the form of a powder, tablet, gel capsule, or liquid immediately after simultaneously dispensing the first and second liquid components.

23. The dental mouthwash product of claim 19 wherein the chlorite compound is present at a concentration in the range of about 0.1 to 0.5% by weight, the H₂O₂ is present at a concentration of about 0.01 to 1.5% by weight, and the oxidizing agent is present at a concentration of about 0.01 to 33% by weight.

24. An aqueous solution comprising about 0.05 to 0.5% ClO₂⁻, about 0.3 to about 1.5% H₂O₂ by weight, about 0.01 to about 33%, by weight, of a physiologically acceptable oxidizing agent that generates ClO₂ from ClO₂⁻ within seconds, and which is stable in the present of 1.5% H₂O₂, and about 1 to 100 ppm ClO₂.

25. The solution of claim 24 wherein the oxidizing agent is any of ascorbic acid, erythorbic acid, tannic acid, folic acid, retinoic acid, sorbic acid, adipic acid, glutathione, t-butylhydroperoxide, and 2,4-hexadienal, or any other physiologically acceptable oxidizing agent that generates ClO₂ from ClO₂⁻ within seconds, and which is stable in the present of 1.5% H₂O₂, or mixtures thereof.

26. The solution of claim 25 wherein the oxidizing agent is a salt of ascorbate.

27. An antimicrobial mouthwash comprising:
a water-soluble chlorite compound;
hydrogen peroxide; and
an oxidizing agent,
wherein the oxidizing agent is stable in the presence of 1.5% H₂O₂, oxidizes chlorite to ClO₂ within seconds, and may be any of ascorbic acid, erythorbic acid, tannic acid, any other physiologically acceptable oxidizing agent that is stable in the presence of 1.5% H₂O₂ and oxidizes chlorite to ClO₂ within seconds, or mixtures thereof.

28. The antimicrobial mouthwash according to claim 27, wherein the water-soluble chlorite compound is present in a first solution at a concentration in the range of about 0.1 to 0.5% by weight, the H₂O₂ is present in a second solution at a concentration of about 1.5% or less, by weight, and the oxidizing agent is optionally present in the second solution at a concentration of about 0.01 to about 33% by weight, or added as a tablet immediately after mixing the first and second solutions.

29. The antimicrobial mouthwash according to claim 28, wherein the oxidizing agent is present in the second solution, and appropriate volumes of the first and second solutions to generate chlorine dioxide are mixed prior to use as a mouthwash.

30. The antimicrobial mouthwash according to claim 28, wherein the oxidizing agent is added in the form of a powder, tablet, gel capsule, or liquid immediately after mixing appropriate volumes of the first and second solutions to generate chlorine dioxide.

31. The antimicrobial mouthwash according to claim 30, wherein the oxidizing agent is added as an effervescent tablet.

32. A method of whitening teeth comprising the steps of:
a) providing a water-soluble chlorite compound in a first solution;
b) providing hydrogen peroxide and an oxidizing agent that is stable in the presence of 1.5% hydrogen peroxide and that oxidizes chlorite to ClO₂ within seconds, the hydrogen peroxide present in a second solution optionally containing the oxidizing agent;
c) mixing the first solution and the second solution and oxidizing agent to produce a mixed composition; and
d) applying the mixed composition of step (c) as a mouthwash on a regular basis.

33. A method of whitening teeth according to claim 32, wherein the oxidizing agent is present in the second solution and is any of ascorbic acid, erythorbic acid, tannic acid, folic acid, retinoic acid, sorbic acid, adipic acid, glutathione, t-butylhydroperoxide, and 2,4-hexadienal or any other physiologically acceptable oxidizing agent that is stable in 1.5% H₂O₂ and oxidizes chlorite to ClO₂ within seconds, and mixtures thereof

34. A method of whitening teeth according to claim 32, wherein the oxidizing agent is added in the form of a powder, tablet, gel capsule or liquid immediately after mixing the first and second solutions, and is any of ascorbic acid, erythorbic acid, tannic acid, folic acid, retinoic acid, sorbic acid, adipic acid, glutathione, t-butylhydroperoxide, and 2,4-hexadienal or any other physiologically acceptable oxidizing agent that is stable in 1.5% H₂O₂ and oxidizes chlorite to ClO₂ within seconds, and mixtures thereof.

35. A method of whitening teeth according to claim 32, wherein the chlorite is present in the first solution at a concentration in the range of about 0.05 to 0.5%, the hydrogen peroxide is present in the second solution at a concentration of about 0.1 to 1.5%, by weight, and the oxidizing agent is present in the mixed composition at a concentration of between about 0.01 to 33% by weight.

36. A method of strengthening gum and ligament attachments to teeth comprising the steps of:
a) providing a water-soluble chlorite compound in a first solution;
b) providing hydrogen peroxide and ascorbic acid, the hydrogen peroxide present in a second solution optionally containing the ascorbic acid;
c) mixing the first solution and the second solution and ascorbic acid to produce a mixed composition; and
d) applying the mixed composition of step (c) as a mouthwash on a regular basis.

37. The method of claim 36, said method having one or more of the following features:
the water-soluble chlorite compound in the first solution is present at a concentration in the range of about 0.25 to 0.5% by weight;
the water-soluble chlorite compound is NaClO₂;
the ascorbic acid is present in the second at a concentration in the range of about 0.01 to 33% by weight;
the ascorbic acid is added immediately after mixing the first and second solutions in the form of a powder, tablet, gel capsule, or liquid;
the hydrogen peroxide in the second solution is present at a concentration in the range of about 0.01 to 1.5% by weight; and
the antimicrobial composition has a pH in the range of 1-8 after mixing.

38. The method of claim 36 wherein:
the water-soluble chlorite compound in the first solution is present at a concentration in the range of about 0.25 to 0.5% by weight;
the water-soluble chlorite compound is NaClO₂;
the ascorbic acid is present in the second solution at a concentration in the range of about 0.01 to 33% by weight;
the hydrogen peroxide in the second solution is present at a concentration in the range of about 0.01 to 1.5% by weight; and
the mixed composition has a pH in the range of 2-8 after mixing.

39. The method of claim 36 wherein:
the water-soluble chlorite compound in the first solution is present at a concentration in the range of about 0.25 to 0.5% by weight;
the water-soluble chlorite compound is NaClO₂;
the ascorbic acid is added immediately after mixing the first and second solutions in the form of a powder, tablet, gel capsule, or liquid to achieve a concentration in the range of about 0.01 to 33% by weight; and
the mixed composition has a pH in the range of 2-8 after mixing.

40. A method of preventing dental caries comprising the steps of:
a) providing a water-soluble chlorite compound in a first solution;
b) providing hydrogen peroxide and ascorbic acid, the hydrogen peroxide present in a second solution optionally containing the ascorbic acid;
c) mixing the first solution and the second solution and ascorbic acid to produce a mixed composition; and
d) applying the mixed composition of step (c) as a mouthwash on a regular basis.

41. The method of claim 40, said method having one or more of the following features:
the water-soluble chlorite compound in the first solution is present at a concentration in the range of about 0.25 to 0.5% by weight;
the water-soluble chlorite compound is NaClO₂;
the ascorbic acid is present in the second at a concentration in the range of about 0.01 to 33% by weight;
the ascorbic acid is added immediately after mixing the first and second solutions in the form of a powder, tablet, gel capsule, or liquid;
the hydrogen peroxide in the second solution is present at a concentration in the range of about 0.01 to 1.5% by weight; and
the antimicrobial composition has a pH in the range of 1-8 after mixing.

42. The method of claim 40 wherein:
the water-soluble chlorite compound in the first solution is present at a concentration in the range of about 0.25 to 0.5% by weight;
the water-soluble chlorite compound is NaClO₂;
the ascorbic acid is present in the second solution at a concentration in the range of about 0.01 to 33% by weight;
the hydrogen peroxide in the second solution is present at a concentration in the range of about 0.01 to 1.5% by weight; and
the mixed composition has a pH in the range of 2-8 after mixing.

43. The method of claim 40 wherein:
the water-soluble chlorite compound in the first solution is present at a concentration in the range of about 0.25 to 0.5% by weight;
the water-soluble chlorite compound is NaClO₂;
the ascorbic acid is added immediately after mixing the first and second solutions in the form of a powder, tablet, gel capsule, or liquid to achieve a concentration in the range of about 0.01 to 33% by weight; and
the mixed composition has a pH in the range of 2-8 after mixing.
